(19) United States
(12) Patent Application Publication
(10) Pub. No.: US 2023/0181359 A1
(43) Pub. Date: Jun. 15, 2023

(54) MITIGATING PRESSURE PULSE IN VACUUM LINE USING A PRESSURE WAVE DAMPER

(71) Applicant: JOHNSON & JOHNSON SURGICAL VISION, INC., Santa Ana, CA (US)

(72) Inventors: Assaf Govari, Haifa (IL); Vadim Gliner, Haifa (IL); Ilya Sitnitsky, Nahariya (IL)

(21) Appl. No.: 17/551,243

(22) Filed: Dec. 15, 2021

(51) Int. Cl. A61F 9/007

(52) U.S. Cl. CPC A61F 9/00745; A61B 2217/005

(57) ABSTRACT

A system for controlling aspiration of a phacoemulsification system having (i) a device that is coupled with an aspiration line of the phacoemulsification system to regulate flow in the aspiration line, and (ii) a pressure wave damper, which is fluidly coupled with the aspiration line and includes an elastic element, the elastic element configured to undergo a change in shape in response to a pressure pulse created by the device, so as to suppress an amplitude of the pressure wave before the pressure pulse reaches the eye.

### FIELD OF THE DISCLOSURE

The present disclosure relates generally to phacoemulsification systems and probes, and particularly to systems for aspiration and irrigation control.

### BACKGROUND OF THE DISCLOSURE

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution (BSS) to maintain the anterior chamber of the eye. After removing the cataract with phacoemulsification, the softer outer lens cortex is removed with suction. An intraocular lens (IOL) is then introduced into the empty lens capsule restoring the patient's vision.

Various techniques of irrigation and aspiration control with medical probes were proposed in the patent literature. For example, U.S. Patent Application Publication 2006/0135974 describes a length of a tubing that includes an irrigation lumen for carrying irrigation fluid from a source to an ophthalmic surgical site at an eye. An aspiration lumen is also formed in a tubing for carrying aspirant from the surgical site at the eye to a collection reservoir. The irrigation lumen and the aspiration lumen include a compliant common wall. Wall ensures that any surge occurring after an occlusion break during surgery is dampened because of the compliant common wall.

As another example, U.S. Pat. No. 5,476,448 describes a surge suppresser which has a collapsible flexible wall that accumulates the aspiration fluid of an interocular surgical system. The surge suppresser has an inlet port and an outlet port that are connected to an aspiration line downstream from a surgical tip. The aspiration line is connected to a vacuum device which draws fluid from the tip. When an occlusion in the aspiration line occurs, the decrease in pressure caused by the vacuum device will cause the flexible wall to collapse and close the inlet and outlet ports of the surge suppresser. When the occlusion breaks, the inlet port initially opens and the outlet port remains closed so that the flexible wall is expanded by the flow of fluid from the surgical tip. The closed outlet port prevents a surge of fluid from the eye. Additionally, the ports are arranged in a parallel relationship to reduce the momentum of the fluid into the suppresser and further limit the surge of fluid from the eye. As the flexible wall gradually expands, the outlet port opens and allows the fluid to flow through the suppresser.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification apparatus comprising an aspiration and irrigation control module, in accordance with an example of the present disclosure;

FIG. 2 is a schematic block diagram of a sleeve pressure wave damper externally coupled to the aspiration and irrigation control module of FIG. 1, in accordance with an example of the present disclosure;

FIG. 3 is a schematic block diagram of a balloon pressure wave damper externally coupled to the aspiration and irrigation control module of FIG. 1, in accordance with an example of the present disclosure;

FIG. 4 is a schematic block diagram of the sleeve pressure wave damper of FIG. 2 and the balloon pressure wave damper of FIG. 3 incorporated into the aspiration and irrigation control module of FIG. 1, in accordance with an example of the present disclosure;

FIG. 5 is a schematic block diagram of a compliant membrane pressure wave damper externally coupled to the aspiration and irrigation control module of FIG. 1, in accordance with an example of the present disclosure; and

FIG. 6 is a flow chart schematically illustrating a method for overcoming a pressure wave caused by a device that regulates flow in the aspiration ways, in accordance with some examples of the present disclosure.

### DETAILED DESCRIPTION OF EXAMPLES

### Overview

During phacoemulsification, emulsified lens particles are aspirated via an aspiration channel of the probe and further proximally into an aspiration line. When a particle blocks the inlet of the aspiration channel the vacuum in the channel and the line increases. When the channel later becomes unblocked (e.g., when the particle is subsequently sucked into the line), the high vacuum in the line causes an aspiration surge with potentially traumatic consequences to the eye.

When a vacuum surge is detected, a possible responsive measure is to close the vacuum line with a valve. However, vacuum buildup downstream of a closed valve may be accompanied, after the valve is reopened, with a positive pressure pulse. The positive pressure pulse may lead to a reverse flow into the eye. The pressure pulse in an aspiration line is extremely narrow in time, typically lasting several tens of milliseconds. Such a pulse may therefore act similarly to a “water hammer” that occurs in household plumbing when a tap closes water flowing from a water pipe that contains air. If such a pressure pulse feeds back to the eye, it can cause trauma. The pulse may also cause material that is held by the tip of the phacoemulsification probe or handpiece to be released and/or repelled from the tip.

A recent solution to the problem of vacuum surge is described in U.S. patent application Ser. No. 17/130409, filed on Dec. 22, 2020, and titled, “A module for Aspiration and Irrigation Control,” whose disclosure is incorporated herein by reference. The application discloses an anti-vacuum surge (AVS) module coupled to a phacoemulsification probe, which uses valves to prevent a sudden vacuum increase from being transferred into the eye when an occlusion breaks. However, even an AVS solution may give rise to the opposite phenomenon of sudden air pressure rising in the aspiration channel that is transferred into the eye.

A vacuum surge can additionally or alternatively be mitigated by, as another example, using a venting valve that lets ambient air flow into the vacuum line. However, using a venting valve to dampen a vacuum surge may also cause the opposite phenomenon of sudden air pressure rising in the line that may cause emulsified material (particles) in the aspiration channel to fly back into the eye with harmful consequences.

Examples of the present disclosure that are described hereinafter incorporate a pressure wave damper (also referred to as a pressure shock absorber) into the aspiration or vacuum line. The pressure damper dampens any pressure wave in case of a sudden rise in pressure caused by a device that is coupled to regulate fluid flow in the aspiration line, thereby creating the pressure pulse. The device may be a valve, a pump, or the aforementioned AVS module, for example. The pressure wave damper is fluidly coupled with the aspiration channel and comprises an elastic element. By undergoing a change in shape in response to a pressure wave, the elastic element of the pressure wave damper suppresses an amplitude of the pressure wave before the pressure wave reaches the eye.

For example, just after the AVS module reverts to its normal ongoing operating format, the pressure wave damper, which is a fully passive element, automatically moderates any resultant pressure transfer (i.e., prevents a pressure overshoot), by ensuring a preconfigured gradual rise rate in pressure to the nominal vacuum level.

Specifically, in case of using an AVS, though a given amount of air still remains in the aspiration line, the AVS module may safely revert to its normal ongoing operating format, so that there is neither an abrupt high vacuum transfer to the eye nor to a pressure wave.

Furthermore, the pressure wave damping solutions described herein can be used with devices other than the AVS, such as those that are located more remotely from the handpiece (e.g., control of an aspiration pump at a console), or others located more distally (e.g., a passive bypass channel), all which may require such protection of the eye from the forementioned pressure pulse propagating towards the eye via aspiration fluid.

The various pressure wave dampers described herein can be located inside or outside the device (e.g., inside or outside the AVS module).

In one example, the pressure wave damper (e.g., the shock absorber) comprises perforations in the line, said perforations enclosed by a compliant tube which seals the perforations and prevents fluid from exiting the line.

When a positive pressure pulse in the line encounters the pressure wave damper, the tube expands outward at its center, while remaining in contact with the line at its edges. The tube thus prevents fluid from leaking from the system. However, the tube does allow a small amount of fluid to temporarily exit from, and return to, the line, thus reducing the pressure in the pulse. The pressure wave damper acts in the same way as a bypass, or smoothing, capacitor in an electrical circuit.

In another example, an air pocket is embedded in a rigid compartment coupled externally to, or included within, the handpiece or the AVS module.

In one example, the air pocket (e.g., a rigid compartment or a cell) is fluidly coupled with the aspiration line by a compliant membrane. As a pressure pulse in the line arrives at the location of the air pocket, the compliant membrane compresses the air in the pocket, allowing some of the fluid in the line to temporarily exit the line. When the pulse passes the location, the air in the pocket decompresses and the fluid returns to the line. The compression and decompression of the air in the pocket, and consequent exit and return of fluid to the aspiration line, reduces the pressure in the pressure pulse.

In another example, the membrane is flexible and fixed at its perimeter, as described below. In yet another example the membrane is stiffer and causes a movable frame that is sealed against a wall of the air pocket, to which the membrane is coupled, to move (i.e., the membrane acts like a “sail”) and thereby compresses the air in the pocket. The air pocket may be a cell, or may comprise an internal air balloon inside the cell.

In an alternative example, rather than having an air pocket connected to the aspiration line by a membrane, a balloon that is surrounded by an internal air jacket is connected to the line. When the pulse passes, the air in the jacket compresses and decompresses to dampen the pressure wave.

In a further example, rather than having an air pocket connected to the aspiration line by a membrane, a moving piston is coupled with the air pocket and moves to compress and decompress the gas in the air pocket to dampen the pressure wave.

In some examples the above-described one or more pressure wave solutions is coupled externally with the AVS module. In other examples, the above-described one or more pressure wave solutions are included in the AVS module.

### System Description

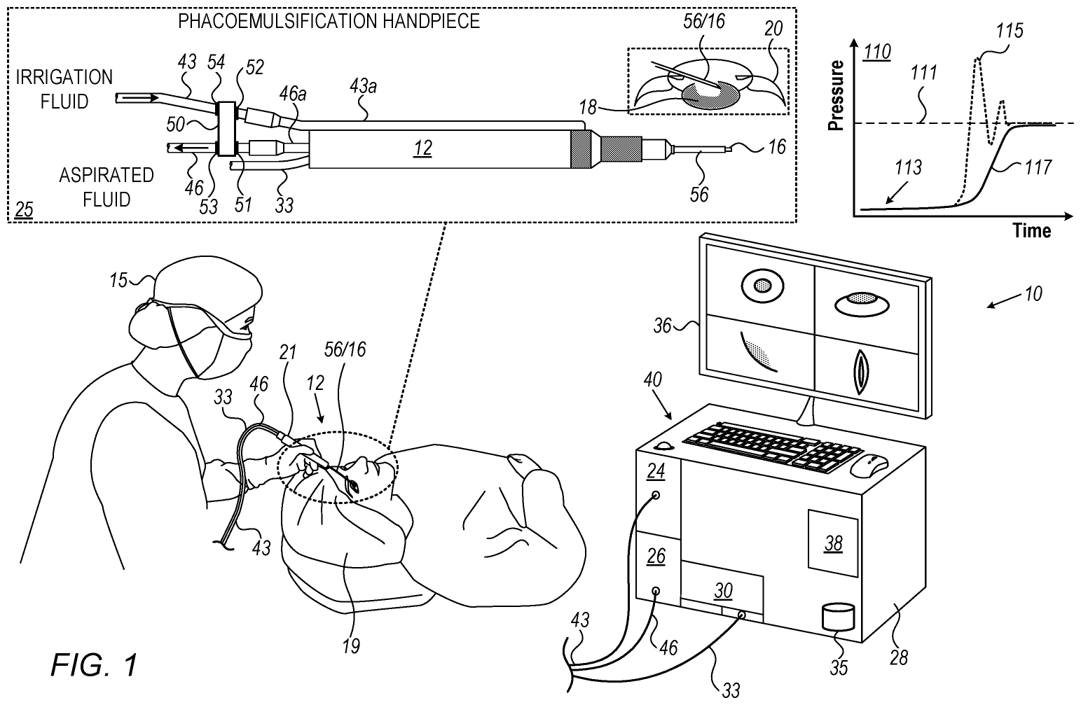

FIG. 1 is a schematic, pictorial view, along with an orthographic side view, of a phacoemulsification apparatus 10 comprising an aspiration and irrigation control module 50 (i.e., a device 50), in accordance with an example of the present disclosure.

As seen in the pictorial view of phacoemulsification apparatus 10, and in inset 25, a phacoemulsification probe 12 (e.g., a handpiece) comprises a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Santa Ana, Calif., USA.

In the shown example, during the phacoemulsification procedure a pumping subsystem 24 comprised in a console 28 pumps irrigation fluid from an irrigation reservoir to the irrigation sleeve 56 to irrigate the eye. The fluid is pumped via an irrigation tubing line 43 running from the console 28 to an irrigation channel 43a of probe 12. Eye fluid and waste matter (e.g., emulsified parts of the cataract) are aspirated via hollow needle 16 to a collection receptacle (not shown) by a pumping subsystem 26, also comprised in console 28, using an aspiration tubing line 46 running from aspiration channel 46a of probe 12 to console 28. In another example, the pumping subsystem 24 may be coupled or replaced with a gravity-fed irrigation source such as a balanced salt solution bottle/bag.

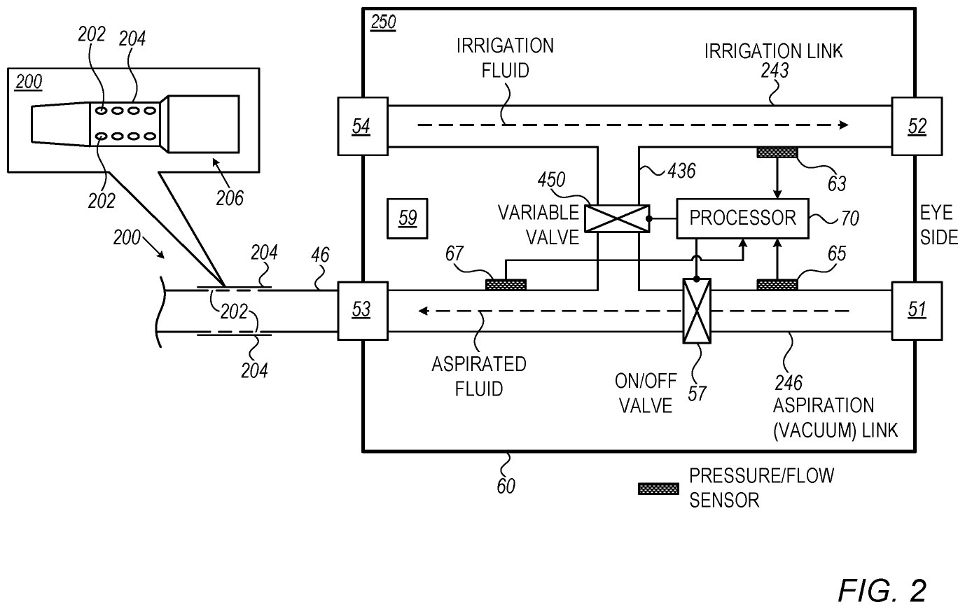

Apparatus 10 includes standalone disposable detachable add-on module 50, also called device 50, (e.g., an AVS module shown in FIGS. 2-5), coupled via fluid connectors 51-54, to control aspiration and irrigation flow rates to reduce risks to eye 20 from irregular performance of aspiration and/or irrigation in probe 12, such as from a vacuum surge. To this end, the disclosed module 50 establishes variable fluid communication between aspiration channel 46a and irrigation channel 43a to control the flow of fluid between the two channels/tubing lines, so as to maintain pressures in the two channels/tubing lines within predefined limits. Moreover, module 50 can discontinue aspiration in parallel in order to provide a fast response (e.g., within several milliseconds) to a detected vacuum surge. Module 50 has its own processor and can be used with existing phacoemulsification systems as a disposable element to improve control over intraocular pressure (IOP) during the surgical cataract removal procedure.

As graph 110 shows, during restoration of vacuum level from a vacuum surge level 113 to a nominal vacuum level 111, a pressure pulse 115, potentially harmful to the eye, may occur. A typical nominal vacuum level is 350 mmHg, whereas a typical peak pressure amplitude of pulse 115 may be in the order of 650 mmHg. The disclosed pressure damping solutions, including those described in FIGS. 2-5, eliminate or reduce the pulse and provide moderated increase of pressure in the general form of pressure curve 117.

Phacoemulsification probe 12 includes other elements (not shown), such as a piezoelectric crystal coupled to a horn to drive vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal, using electrical wiring running in a cable 33. Drive module 30 is controlled by a processor 38 and conveys processor-controlled driving signals via cable 33 to, for example, maintain needle 16 at maximal vibration amplitude. The drive module may be realized in hardware or software, for example, in a proportional-integral-derivative (PID) control architecture.

Processor 38 may receive user-based commands via a user interface 40, which may include setting a vibration mode, duty cycle, and/or frequency of the piezoelectric crystal, and setting or adjusting an irrigation and/or aspiration rate of the pumping subsystems 24/26. In an example, user interface 40 and display 36 may be combined as a single touch screen graphical user interface. In an example, the physician uses a foot pedal (not shown) as a means of control. Additionally or alternatively, processor 38 may receive the user-based commands from controls located in a handle 21 of probe 12.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively or additionally, the software may be stored in tangible, non-transitory computer-readable storage media, such as optical, magnetic, or electronic memory.

The apparatus shown in FIG. 1 may include further elements which are omitted for clarity of presentation. For example, physician 15 typically performs the procedure using a stereomicroscope or magnifying glasses, neither of which are shown. Physician 15 may use other surgical tools in addition to probe 12, which are also not shown in order to maintain clarity and simplicity of presentation.

In some examples, a different type of AVS module can be used that is coupled only to the aspiration part of the system (i.e., without involving irrigation).

### Pressure Wave Damper for Vacuum Line with AVS Module

As noted above, a challenge may exist with different types of aspiration and irrigation control solutions to prevent an occurrence of a stray pressure wave that can harm the eye during restoration of nominal aspiration performance. The examples shown in FIGS. 2-5 are of a pressure wave damping solution used in conjunction with an AVS module. However, the disclosed solution may be incorporated with other aspiration and irrigation control schemes known in the art.

FIG. 2 is a schematic block diagram of a sleeve pressure wave damper 200 externally coupled with the module of FIG. 1, which herein is an AVS module 250, in accordance with an example of the present disclosure.

In the shown example, standalone module 250 includes a battery 59 inside a package 60 of module 250 to power a processor, sensors, and electromechanical valves.

As seen, package 60 includes connectors 51-54 fitted on the package 60 that are configured to couple the aspiration and irrigation channels of a probe (46a and 43a via connectors 51 and 52, respectively), and to couple the respective aspiration and irrigation lines of the phacoemulsification system (46 and 43 via connectors 53 and 54, respectively) to the module 250.

Inside package 60 there is an irrigation link 243 to flow irrigation fluid from line 43 into irrigation channel 43a, and an aspiration link 246 to remove material from aspiration channel 46a into aspiration line 46.

Furthermore, irrigation link 243 is fluidly coupled with aspiration link 246 via a bypass channel 436. As seen, a diversion (processor-controlled) variable valve 450 on bypass channel 246 is configured to control a level of fluid communication between irrigation link 243 and aspiration link 246.

A processor-controlled aspiration valve 57 is configured to open or close aspiration link 246 at a distal portion thereof, to, for example, immediately suppress a vacuum surge.

To provide feedback, a sensor 63, such as a pressure sensor or a flow sensor, is coupled with irrigation link 243 to measure the irrigation fluid parameters (e.g., pressure or flow rate) in irrigation link 243 distally to bypass channel 436. A sensor 65 (such as a pressure sensor or a vacuum sensor) similarly measures the aspiration pressure/vacuum in aspiration link 246 distally to bypass channel 436. An additional sensor 67 similarly measures the flow/pressure/vacuum in aspiration link 246 proximally to bypass channel 436. The pressure/flow measurements of the irrigation link 243 and pressure/vacuum measurements of the aspiration link 246 are performed close to irrigation outlet 52 and aspiration inlet 51, respectively, so as to provide an accurate indication of the actual pressures experienced by an eye and provide quick response time to a control loop of module 250.

Based on the fluid pressure/flow measured by sensors 63-67, a processor 70 included in module 250 adaptively adjusts an opening of bypass channel 436 by adjusting variable valve 450, and, in coordination, closes or opens aspiration valve 57.

In some examples, a different type of AVS module can be used that is coupled only with the aspiration part of the system (i.e., without involving irrigation). In such an AVS module there is no diversion of irrigation, and aspiration flow is solely determined by elements on the aspiration line/channel, such as the aspiration pump and valve 57.

In the example shown in FIG. 2, sleeve pressure wave damper 200 (e.g., shock absorber) comprises perforations 202 in line 46, and perforations 202 are enclosed by a compliant tube 204 which seals the perforations and prevents fluid from exiting the line. In practice, for example for modularity, line 46 may be fitted with connectable wave damper 200. As seen, wave damper 200 has perforations 202 in a rigid tube 206 of wave damper 200 and compliant tube 204 encloses such perforated portion of rigid tube 206.

When a positive pressure pulse in the line encounters sleeve pressure wave damper 200, compliant tube 204 (also called elastic sleeve 204), which may be made, for example, from an elastic polymer, expands outward at its center, while remaining in contact with rigid tube 206 at its edges. The compliant tube 204 thus prevents fluid from leaking from the system. However, the compliant tube 204 does allow a small amount of fluid to temporarily exit the aspiration line 46, and then return to the aspiration line 46. The temporary fluid exit and return thereby reduces the pressure in the pulse. The moderation is determined by the geometry of wave damper 200 and by the elasticity of compliant tube 204, and a proper design can eliminate the occurrence of any pressure pulse.

The example shown in FIG. 2 is chosen purely for the sake of conceptual clarity. For example, other designs of sleeve pressure wave damper 200 may include different perforation geometries, and different compliant tube materials, such as those that are partially porous.

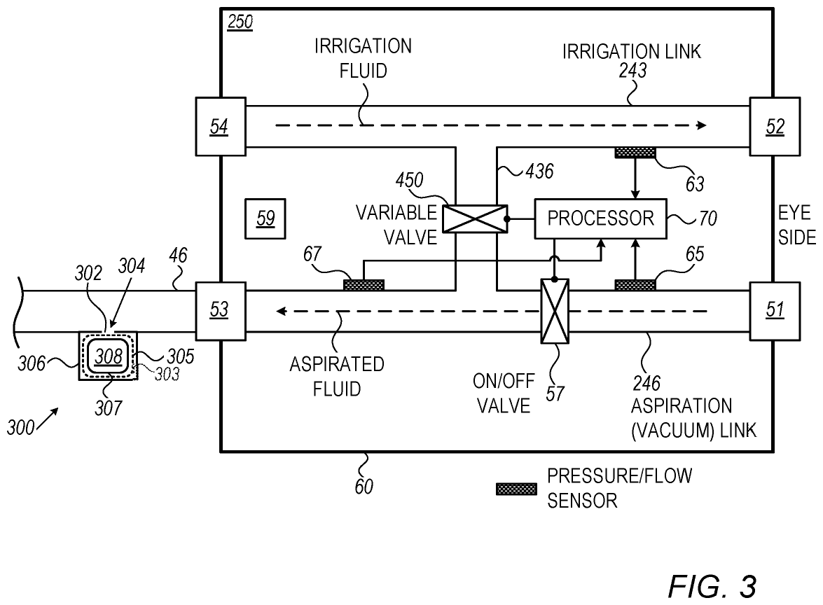

FIG. 3 is a schematic block diagram of a balloon pressure wave damper 300 externally coupled with aspiration and irrigation control module of FIG. 1, which herein is an AVS module 250, in accordance with an example of the present disclosure. Elements of module 250 are described in FIG. 2.

In the shown example, a compartment 306 has a “double wall” structure with an internal, perforated, wall 303. The perforated internal wall 303 creates an internal air jacket 304 (the inside volume of the double wall) that is a medium 302 fluidly coupled with the aspiration line 46 since compartment 306 (e.g., air pocket 306) is fluidly connected with the aspiration line. The internal perforated wall 303 holds inside a balloon 307 surrounded by internal air jacket 304. When the pulse passes, the air in jacket 304 compresses and decompresses at a given rate determined by balloon 307 (the internal air jacket 304 and the balloon are fluidly-coupled via perforations 305) to dampen the pressure wave. The rate at which nominal vacuum is restored, as seen by curve 117 of FIG. 1, is determined by, among other factors, the volumes of the elements and the pressure of gas 308 inside balloon 307. By way of example, to restore a nominal vacuum level 111 (i.e., sub-pressure) of about 50 millibar without an overshooting wave amplitude 115, gas 308 pressure is preset to about 25 millibar.

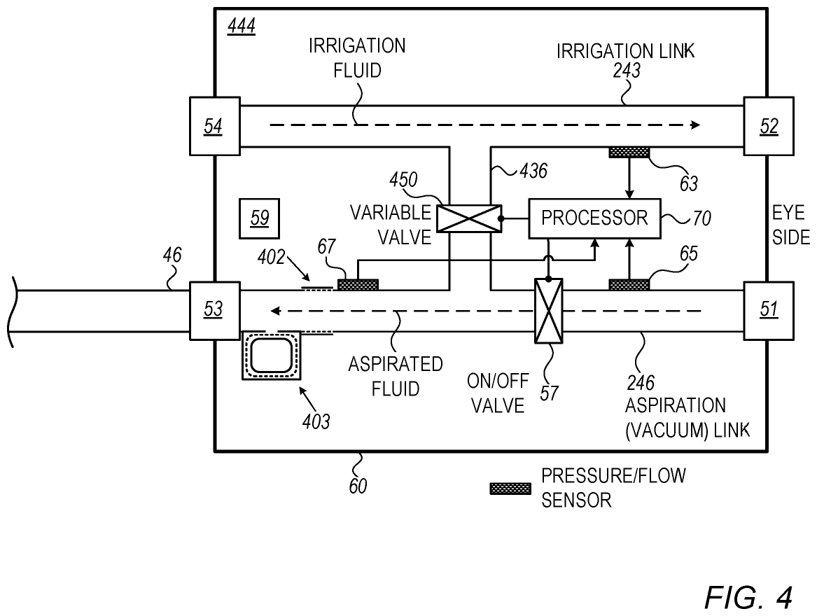

FIG. 4 is a schematic block diagram of the sleeve pressure wave damper 200 of FIG. 2 (herein numbered 402) and the balloon pressure wave damper 300 (herein numbered 403) of FIG. 3 incorporated into an aspiration and irrigation control module 444, such as AVS module 250 of FIGS. 2 and 3, in accordance with an example of the present disclosure. By including sleeve pressure wave pressure damper 402 and/or balloon pressure wave damper 403 in AVS module 444, it becomes a full standalone solution, e.g., one also capable of suppressing a post-occlusion vacuum surge pressure pulse. Elements of module 444 are described in FIG. 2.

As noted above, in some examples a different type of AVS module may be used that is coupled only with the aspiration part of the system (i.e., without involving irrigation). In such an AVS module there is no diversion of irrigation, and aspiration flow is solely determined by elements on the aspiration line/channel, such as the aspiration pump and valve 57, and pressure dampers 402 and/or 403.

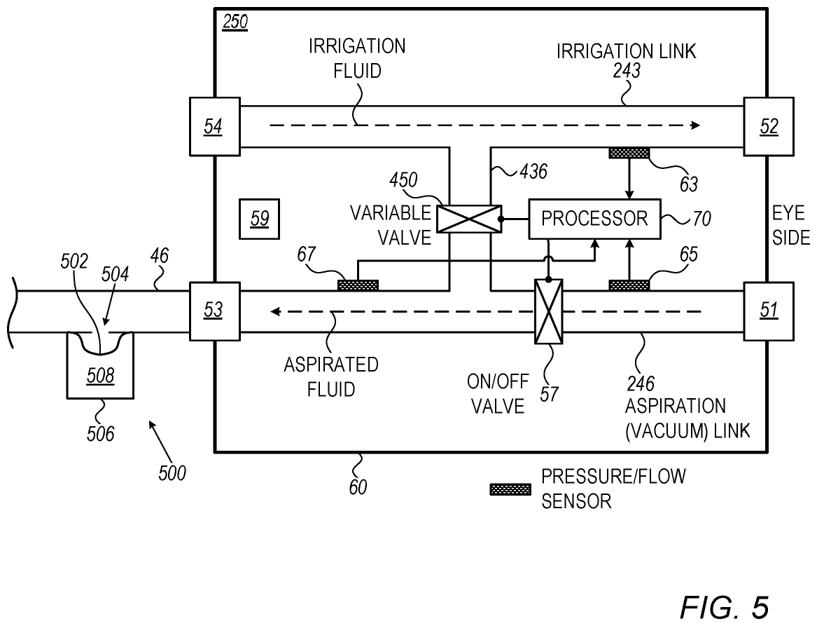

As noted above a pressure wave damper may have numerous realizations. FIG. 5 is a schematic block diagram of a compliant membrane pressure wave damper 500 externally coupled with aspiration and irrigation control module of FIG. 1, which herein is an AVS module 250, in accordance with an example of the present disclosure. Elements of module 250 are described in FIG. 2.

In the shown example, an air pocket 506 is fluidly connected (504) with the aspiration line 46. Air pocket (e.g., compartment) 506 includes a compliant membrane 502. When a pressure pulse in the aspiration line 46 arrives at the location of the air pocket 506, compliant membrane 502 compresses the air 508 in the air pocket 506 allowing some of the fluid in the aspiration line 46 to temporarily exit the aspiration line. When the pulse passes the location, air 508 in the air pocket 506 decompresses and the fluid returns to the aspiration line 46. The compression and decompression of the air in the pocket 506, and consequent exit and return of fluid to aspiration line 46, reduces the pressure in the pressure pulse.

The rate at which nominal vacuum is restored, as seen by curve 117 of FIG. 1, is determined by, among other factors, the volumes and pressure of gas 508 behind membrane 502. By way of example, to restore a nominal vacuum level 111 (i.e., sub-pressure) of about 50 millibar without overshooting 115, gas 508 pressure is preset to about 25 millibar.

### Method of Damping a Pressure Wave Caused by a Device that Regulates Flow in the Spiration Ways

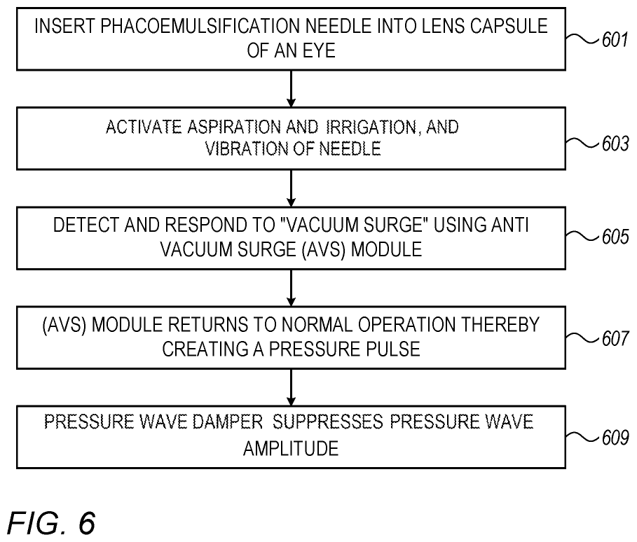

FIG. 6 is a flow chart schematically illustrating a method for overcoming a pressure pulse(s) caused by a device that regulates flow in the aspiration line, in accordance with some examples of the present disclosure. The process begins with physician 15 inserting phacoemulsification needle 16 of probe 12 into a lens capsule 18 of an eye 20, at a phacoemulsification needle insertion step 601.

At a phacoemulsification step 603, physician 15 presses a foot pedal to a first position to activate irrigation and subsequently to a second position to activate aspiration, and finally, when the foot pedal is pressed and placed in a third position, the needle 16 is vibrated to perform the phacoemulsification procedure.

During phacoemulsification, AVS module 250 may overcome vacuum surge (e.g., by closing valve 57) in a vacuum surge response step 605.

After the AVS module is operated in step 605, a return to normal operation (e.g., opening valve 57) may create a pressure pulse in the aspiration line, that may travel to the eye, at a return to normal regulation step 607.

At a pressure wave damping step 609, the pressure wave damper dampens the resulting pressure pulse amplitude, thereby preventing potential damage to the eye.

### Example 1

A system for controlling aspiration of a phacoemulsification system (10), the system including (i) a device (50) that is coupled with an aspiration line (46) of the phacoemulsification system (10) to regulate flow in the aspiration line (46), and (ii) a pressure wave damper, which is fluidly coupled with the aspiration line (46) and comprises an elastic element, the elastic element configured to undergo a change in shape in response to a pressure pulse created by the device, so as to suppress an amplitude of the pressure wave before the pressure pulse reaches the eye (20).

### Example 2

The system according to example 1, wherein the pressure wave damper is physically connected with the aspiration line (46), separately from the device (50).

### Example 3

The system according to example 1, wherein the pressure wave damper is physically contained in a handpiece (12) of the phacoemulsification system (10).

### Example 4

The system according to example 1, wherein the pressure wave damper is physically contained in the device.

### Example 5

The system according to example 1, wherein the device (50) is an anti-vacuum surge (AVS) module (250), which is coupled between a distal portion of the aspiration line (46) and an aspiration channel (46a) of the handpiece (12) and is configured to mitigate vacuum surges in the aspiration line (46) or aspiration channel (46a) by regulating flow via the aspiration line or aspiration channel.

### Example 6

The system according to any one of examples 1 through 5, wherein the pressure wave damper incudes a perforated portion of a rigid tube (206) surrounded by an elastic sleeve (204) that serves as the elastic element.

### Example 7

The system according to any one of examples 1 through 5, wherein the pressure wave damper is physically contained in the device (50).

### Example 8

The system according to example 1, wherein the pressure wave damper includes an air pocket (506) at a preset air pressure fluidly coupled with the aspiration line (46) by a compliant membrane (502) that serves as the elastic element.

### Example 9

The system according to example 8, wherein the pressure wave damper is physically contained in the device (50).

### Example 10

The system according to example 1, wherein the pressure wave damper includes a balloon (307) inflated with a preset air pressure, the balloon serving as the elastic element, with the balloon surrounded by a rigid frame that creates a surrounding air jacket (304) that is fluidly coupled with the aspiration line (46).

### Example 11

The system according to example 10, wherein the pressure wave damper is physically contained in the device (50).

### Example 12

A method includes coupling a device (50) with an aspiration line (46) of a phacoemulsification system (10) to regulate flow in the aspiration line. A pressure wave damper is fluidly coupled with the aspiration line (46), the damper comprising an elastic element, the elastic element configured to undergo a change in shape in response to a pressure pulse created by the device, so as to suppress an amplitude of the pressure wave before the pressure pulse reaches the eye (20).

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for controlling aspiration of a phacoemulsification system, the system comprising:
a device that is coupled with an aspiration line of the phacoemulsification system to regulate flow in the aspiration line; and
a pressure wave damper, which is fluidly coupled with the aspiration line and comprises an elastic element, the elastic element configured to undergo a change in shape in response to a pressure pulse created by the device, so as to suppress an amplitude of the pressure wave before the pressure pulse reaches the eye.

2. The system according to claim 1, wherein the pressure wave damper is physically connected with the aspiration line, separately from the device.

3. The system according to claim 1, wherein the pressure wave damper is physically contained in a handpiece of the phacoemulsification system.

4. The system according to claim 1, wherein the pressure wave damper is physically contained in the device.

5. The system according to claim 1, wherein the device is an anti-vacuum surge (AVS) module, which is coupled between a distal portion of the aspiration line and an aspiration channel of a handpiece and is configured to mitigate vacuum surges in the aspiration line or aspiration channel by regulating flow via the aspiration line or aspiration channel.

6. The system according to claim 1, wherein the pressure wave damper comprises a perforated rigid tube surrounded by an elastic sleeve that serves as the elastic element.

7. The system according to claim 6, wherein the pressure wave damper is physically contained in the device.

8. The system according to claim 1, wherein the pressure wave damper comprises an air pocket at a preset air pressure fluidly coupled with the aspiration line by a compliant membrane that serves as the elastic element.

9. The system according to claim 8, wherein the pressure wave damper is physically contained in the device.

10. The system according to claim 1, wherein the pressure wave damper comprises a balloon inflated with a preset air pressure, the balloon serving as the elastic element, with the balloon surrounded by a rigid frame that creates a surrounding air jacket that is fluidly coupled with the aspiration line.

11. The system according to claim 10, wherein the pressure wave damper is physically contained in the device.

12. A method, comprising:
coupling a device with an aspiration line of a phacoemulsification system to regulate flow in the aspiration line; and
fluidly coupling a pressure wave damper with the aspiration line, the damper comprising an elastic element, the elastic element configured to undergo a change in shape in response to a pressure pulse created by the device, so as to suppress an amplitude of the pressure wave before the pressure pulse reaches the eye.

13. The method according to claim 12, wherein the pressure wave damper is physically connected with the aspiration line, separately from the device.

14. The method according to claim 12, wherein the pressure wave damper is physically contained in a handpiece of the phacoemulsification system.

15. The method according to claim 12, wherein the pressure wave damper is physically contained in the device.

16. The method according to claim 12, wherein the device is an anti-vacuum surge (AVS) module, which is coupled between a distal portion of the aspiration line and an aspiration channel of a handpiece and is configured to mitigate vacuum surges in the aspiration line or aspiration channel by regulating flow via the aspiration line or aspiration channel.

17. The method according to claim 12, wherein the pressure wave damper comprises a perforated rigid tube surrounded by an elastic sleeve that serves as the elastic element.

18. The method according to claim 17, wherein the pressure wave damper is physically contained in the device.

19. The method according to claim 12, wherein the pressure wave damper comprises an air pocket at a preset air pressure fluidly coupled with the aspiration line by a compliant membrane that serves as the elastic element.

20. The method according to claim 19, wherein the pressure wave damper is physically contained in the device.

21. The method according to claim 12, wherein the pressure wave damper comprises a balloon inflated with a preset air pressure, the balloon serving as the elastic element, with the balloon surrounded by a rigid frame that creates a surrounding air jacket that is fluidly coupled with the aspiration line.

22. The method according to claim 21, wherein the pressure wave damper is physically contained in the device.
